(19) United States
(12) Patent Application Publication
(10) Pub. No.: US 2011/0172746 A1
(43) Pub. Date: Jul. 14, 2011

(54) High Level Laser Therapy Apparatus and Methods

(76) Inventor: Roger Porter, Myrtle Beach, SC (US)

(21) Appl. No.: 13/005,179

(22) Filed: Jan. 12, 2011

Related U.S. Application Data
(60) Provisional application No. 61/294,250, filed on Jan. 12, 2010.

(51) Int. Cl. A61N 5/067

(52) U.S. Cl. 607/89

(57) ABSTRACT

Non-ablative laser treatment apparatus and methods for treating various patient conditions are described. A laser therapy apparatus includes an elongated base, a patient support surface attached to the base, one or more laser devices, and a treatment frame for projecting a collimated laser beam from the one or more laser devices onto a patient positioned on the patient support surface. The treatment frame is movable relative to the base along a longitudinal direction defined by the base. One or more collimators are attached to the treatment frame and each is in optical communication with a respective laser device. Each collimator is configured to receive a laser beam from the respective laser device, collimate the laser beam to a particular cross-sectional area while maintaining the laser beam as generally coherent and monochromatic, and project the collimated laser beam onto the skin of a patient positioned on the patient support surface.

### RELATED APPLICATION

This application claims the benefit of and priority to U.S. Provisional Patent Application No. 61/294,250, filed Jan. 12, 2010, the disclosure of which is incorporated herein by reference as if set forth in its entirety.

### FIELD OF THE INVENTION

The present invention relates generally to laser therapy and, more particularly, to laser therapy apparatus and methods.

### BACKGROUND

Lasers are used for various types of medical treatment. For example, “hot” lasers are typically used in surgery, “mid- or low energy” lasers may be used in photodynamic therapy. Low energy lasers (also called low-level laser therapy) generally deliver significantly less energy to tissue than surgical lasers and mid-power lasers.

During conventional low-level or high-level laser therapy, a physician moves a laser apparatus by hand along the tissue to be treated. The treatment dose from such a device is typically set in advance. Conventional apparatus include operational parameters, such as power level, energy, pulsation rate, per session treatment dose, energy intensity, power density, spot size and exposure time.

### SUMMARY

It should be appreciated that this Summary is provided to introduce a selection of concepts in a simplified form, the concepts being further described below in the Detailed Description. This Summary is not intended to identify key features or essential features of this disclosure, nor is it intended to limit the scope of the invention.

Embodiments of the present invention provide non-ablative laser treatment apparatus and methods for treating various patient conditions, such as musculoskeletal conditions, fibromyalgia, back pain, decubitis ulcers (bed sores), etc. According to some embodiments of the present invention, a laser therapy apparatus includes an elongated base, a patient support surface attached to the base, one or more laser devices, and a treatment frame for projecting a collimated laser beam from each of the one or more laser devices onto a patient positioned on the patient support surface. The base includes opposite elongated side walls and opposite end walls that define a cavity. The treatment frame is secured to the base via a pair of guides secured to the base within the cavity. The treatment frame has an arcuate configuration that extends from one side wall to the other side wall and that resembles a tunnel in which a patient is positioned.

The treatment frame is movable relative to the base along a longitudinal direction defined by the base. The treatment frame extends over the patient support surface and is configured to pass over a patient positioned on the patient support surface. In some embodiments the treatment frame includes a compartment positioned beneath the patient support surface that houses the laser device(s). As such, the laser device(s) moves with the treatment frame during a treatment session.

The apparatus includes a drive system located within the base cavity that is configured to move the treatment frame relative to the base along the longitudinal direction. In some embodiments, the drive system includes an elongated threaded drive shaft that extends between the opposite end walls of the base, a motor operably connected to the drive shaft that is configured to rotate the drive shaft, and a gear attached to the treatment frame that is intermeshed with the threaded drive shaft for producing linear motion of the treatment frame along the longitudinal direction upon rotation of the drive shaft. The drive system is configured to move the treatment frame at a constant speed within a range of between about 0.05 inch per second and about one inch per second.

One or more collimators are attached to the treatment frame and each is in optical communication with a respective laser device. Each collimator is configured to receive a laser beam from the respective laser device, collimate the laser beam to a particular cross-sectional area (e.g., >10 cm², such as between about 12 cm² and about 40 cm², typically about 28 cm²) while maintaining the laser beam as generally coherent and monochromatic, and project the collimated laser beam onto the skin of a patient positioned on the patient support surface, typically in continuous wave (CW) mode and in long pulse pulses (e.g., pulse duration times of 1 minute, 2 minutes, 5 minutes, 10 minutes, etc.).

In some embodiments, each laser device is a neodymium doped yttrium-aluminum-garnet (“Nd:Yag”) laser configured to deliver a laser beam at a wavelength of between about 1064 and 1400 nanometers (nm), and having a power density of about 600 mW/cm². In some embodiments, a visible marker beam source is in optical communication with each collimator. Each collimator is configured to project a visible marker beam generated by the marker beam source, along with the laser beam, onto the skin of a patient positioned on the patient support surface that indicates the location of the collimated laser beam on the skin of the patient.

According to some embodiments of the present invention, the treatment frame includes one or more heat sources configured to warm a patient positioned on the patient support surface. In some embodiments, the heat sources are a plurality of heat lamps.

According to some embodiments of the present invention, a method of treating selected tissue of a patient includes moving a treatment frame relative to a base upon which a patient is supported at a substantially constant speed (e.g., between about 0.05 inch per second and about one inch per second), and projecting a collimated laser beam (e.g., having a wavelength of between about 1064 and 1400 nanometers) with a cross-sectional area of at least 10 cm² and a power density of about 600 mW/cm² from the treatment frame onto the skin of the patient such that selected tissue is exposed to a laser light having an energy delivery rate of at least 420 Joules/min during a treatment session, for a total of at least 1,500 Joules per treatment session, typically 10,000 Joules-20,000 Joules, per treatment session. In some embodiments, the patient is warmed during treatment via at least one heat source on the treatment frame. In some embodiments, the collimated laser beam projected onto the patient has a cross-sectional area of at least about 28 cm².

Selected tissue that may be treated via apparatus and methods of the present invention includes: tissue physiologically linked to peripheral neuropathy, reflex sympathetic dystrophy, trigeminal neuralgia, migraine headaches, stroke, concussions, plantar fascititis, radiculophthy, peripheral neuropathy, sciatica, traumatic nerve injury, diabetic nerve, or restless leg syndrome; tissue physiologically linked to post-operative healing, decubitis wound sores, burns, stasis ulcers, allergic rashes, or insect bites; tissue physiologically linked to spinal pain from herniated disc, spinal pain from herniated bulging disc, back pain from musculoskeletal strain, reflex symphatic dystrophy, or fibromyalgia; tissue physiologically linked to herpes, acquired immune deficiency syndrome (AIDS), multiple sclerosis, psoriasis, rheumatoid diseases, chronic fatigue syndrome, Parkinson's disease, or lupus; tissue physiologically linked to fibromyalgia or costochondritis; tissue physiologically linked to stroke, closed head injury, or spinal cord injury; tissue physiologically linked to coronary artery disease or peripheral vascular disease; tissue physiologically linked to viral flu syndromes or autoimmune diseases; tissue physiologically linked to diabetes or ALS; musculo-skeletal tissue; neurological tissue; wound tissue; and spinal tissue or spinal fluid.

Laser treatment via the laser therapy apparatus of the present invention may have the capacity to bypass the melanin of the skin as well as water and other superficial tissue, penetrating to deeper tissues. Molecules within cells may have an attraction for the energy produced by the laser devices of the present invention. The absorption of the photons of energy by the mitochondria of the cell “power plant” may result in an increased cellular energy level. This increase in activity at the cellular level may result in increased circulation as well as alteration of the conduction of pain signals. It also may lead to a decrease in inflammation and swelling, which may result in a decrease of pain, and an increased rate of healing.

In addition, mitochondria energized by a laser device may provide energy to the cell itself. Millions of energized cells in the local area may break down and eliminate inflammation that is the source of most of a patient's pain. In addition, this energy may stimulate blood flow, and therefore, oxygen, to the area. It also may increase the movement of excess edema away from the area.

Another set of tissue that absorbs the energy of the laser device(s) of the laser therapy apparatus are fibers in the nervous system that transmit messages from the painful area to the brain. These chronic pain signals are transmitted through an entirely different system than the signals of acute or sudden pain. The laser treatment may alter the transmission of pain along these pathways, and the sensitivity of these fibers may be “reset” back to a healthy, normal state.

It is noted that aspects of the invention described with respect to one embodiment may be incorporated in a different embodiment although not specifically described relative thereto. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner. These and other objects and/or aspects of the present invention are explained in detail in the specification set forth below.

### BRIEF DESCRIPTION OF THE DRAWINGS

FIG. 1 is an end perspective view of a laser therapy apparatus looking from the foot end of the apparatus, according to some embodiments of the present invention.

FIG. 2 illustrates the laser therapy apparatus of FIG. 1 with the mattress removed to expose the underlying patient support surface.

FIG. 3 is a side perspective view of the laser therapy apparatus of FIG. 2 with the patient support surface removed to expose the base and cavity, and with an outer cover removed from the treatment frame to expose heat lamps and laser collimators, according to some embodiments of the present invention.

FIG. 4 is a top partial perspective view of the laser therapy apparatus of FIG. 3 illustrating the treatment frame guides and drive system, according to some embodiments of the present invention.

FIG. 5 is an enlarged partial view of the drive system shown in FIG. 4 which illustrates the drive system motor assembly and one end of the drive shaft operably connected thereto, according to some embodiments of the present invention.

FIG. 6 illustrates the drive system bearing housing and an opposite end of the drive shaft rotatably connected thereto, according to some embodiments of the present invention.

FIG. 7 is a partial top perspective view of the base cavity illustrating the adjacent sections of the laser housing compartment, according to some embodiments of the present invention.

FIG. 8 is a partial side perspective view of the treatment frame illustrating the heat lamps and laser collimator secured to the treatment frame wall rear surface, according to some embodiments of the present invention.

FIG. 9 is a partial perspective view of the treatment frame illustrating the laser collimators extending down from the treatment frame wall front surface, according to some embodiments of the present invention.

FIG. 10 is a partial side perspective view of the treatment frame illustrating a laser collimator secured to the treatment frame wall rear surface, according to some embodiments of the present invention.

FIG. 11 is a perspective view of the laser therapy apparatus looking from the head end thereof and illustrating the control panel on the treatment frame, according to some embodiments of the present invention.

### DETAILED DESCRIPTION

The present invention will now be described more fully hereinafter with reference to the accompanying figures, in which embodiments of the invention are shown. This invention may, however, be embodied in many different forms and should not be construed as limited to the embodiments set forth herein. Like numbers refer to like elements throughout. In the figures, certain layers, components or features may be exaggerated for clarity, and broken lines illustrate optional features or operations unless specified otherwise. In addition, the sequence of operations (or steps) is not limited to the order presented in the figures and/or claims unless specifically indicated otherwise. Features described with respect to one figure or embodiment can be associated with another embodiment of figure although not specifically described or shown as such.

It will be understood that when a feature or element is referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other element or intervening elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

It will be understood that although the terms first and second are used herein to describe various features/elements, these features/elements should not be limited by these terms. These terms are only used to distinguish one feature/element from another feature/element. Thus, a first feature/element discussed below could be termed a second feature/element, and similarly, a second feature/element discussed below could be termed a first feature/element without departing from the teachings of the present invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

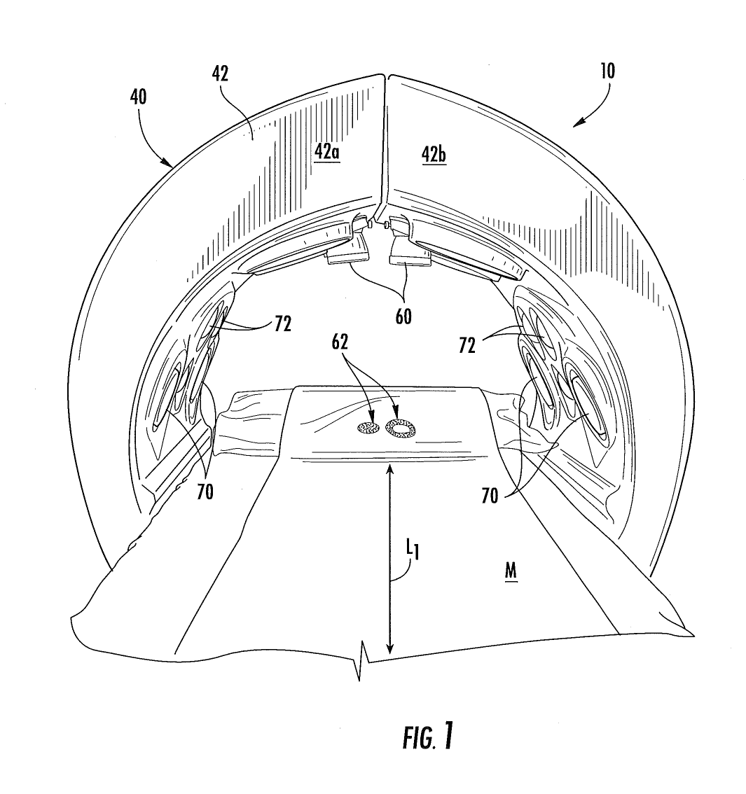

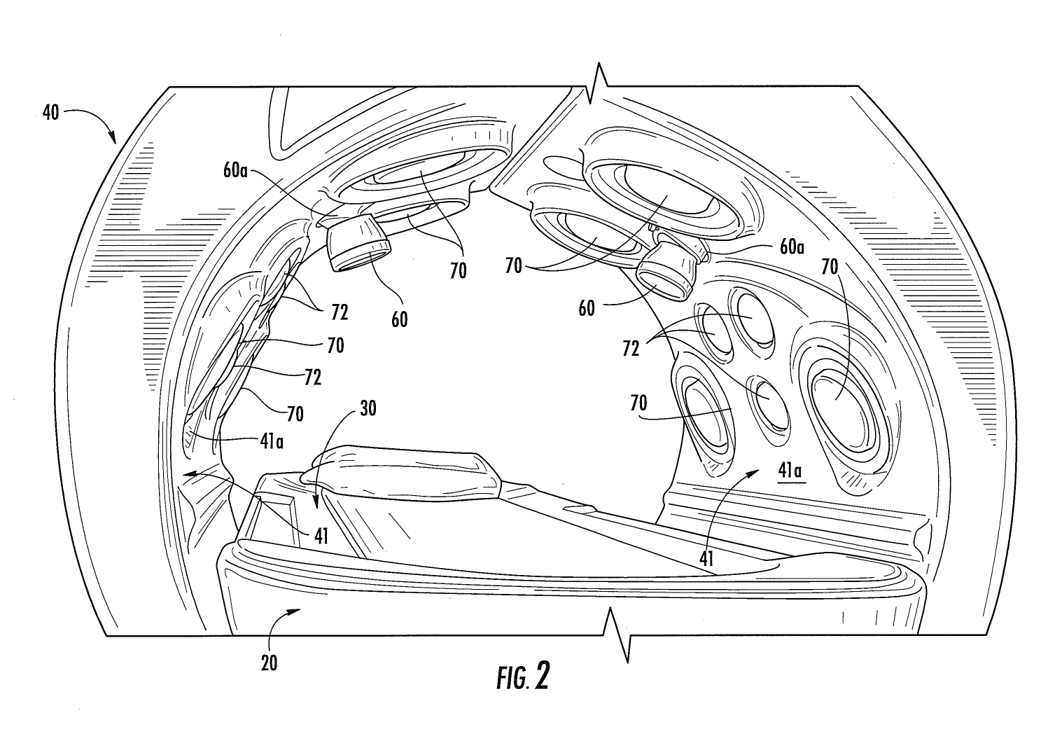

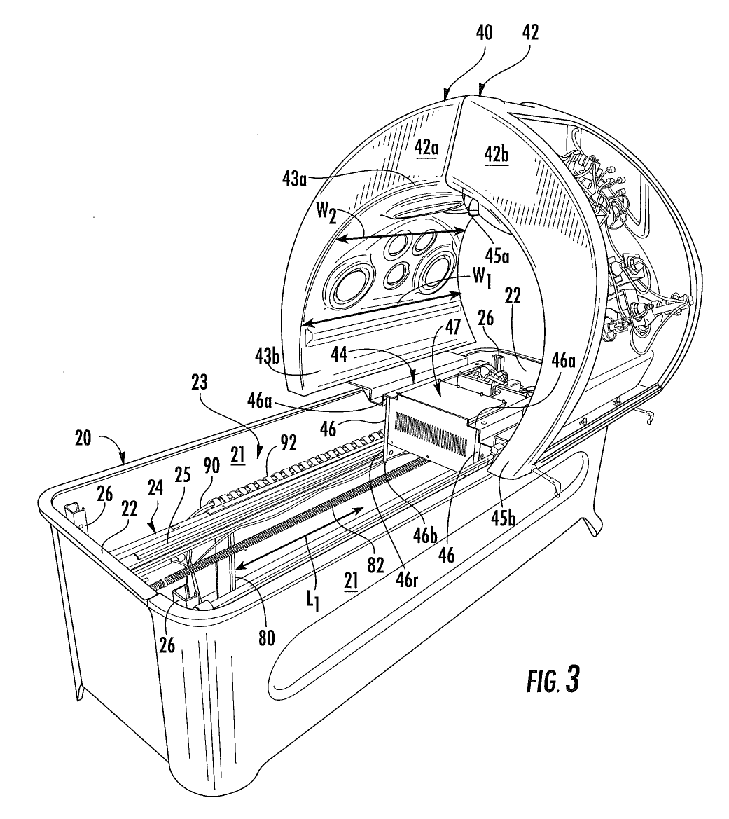

Referring now to FIGS. 1-11, a laser therapy apparatus 10, according to some embodiments of the present invention, is illustrated. The apparatus 10 includes an elongated base 20 (FIG. 3) that defines a longitudinal direction L₁, a patient support surface 30 (FIG. 2) attached to the base 20, and a treatment frame 40 movably secured to the base 20. The treatment frame 40 extends over a portion of the patient support surface 30 and is configured to pass over a target area of a patient positioned on the patient support surface 30 during a treatment session. A mattress M (FIG. 1) is typically positioned on the patient support surface 30 to provide comfort to a patient positioned thereon.

As illustrated in FIG. 3, the base 20 includes opposite elongated side walls 21 and opposite end walls 22 that define a cavity 23. The base 20 also includes a frame 24 positioned within the cavity 23 that is secured to the side walls 21 and end walls 22. Frame 24 is configured to support a pair of guides 25 to which the treatment frame 40 is movably secured. The frame 24 also includes support posts 26 to which the patient support surface 30 is secured.

Still referring to FIG. 3, the treatment frame 40 has an upper portion 42 and a lower portion 44. The upper portion 42 includes a pair of arcuate sections 42a, 42b having respective end portions 43a, 43b and 45a, 45b. The pair of arcuate sections 42a, 42b are joined together at respective end portions 43a, 45a, as illustrated. End portions 43b, 45b are secured to respective leg members 46. In the illustrated embodiment, arcuate section end portions 43b, 45b are secured to upper end portions 46a of the leg members 46. The lower end portions 46b of the leg members 46 include rollers 46r that are movably secured within respective guides 25 and that allow the treatment frame 40 to move relative to the base 20 along the longitudinal direction L₁. Typically, the base 20 is stationary and the treatment frame 40 moves along the longitudinal direction L₁. However, in some embodiments, the treatment frame 40 may be stationary and the base 20 moves such that there is relative movement between the treatment frame 40 and base 20.

The arcuate sections 42a, 42b give the treatment frame 40 an arcuate configuration similar to that of a tunnel that surrounds a portion of a patient positioned on the patient support surface 30. Each arcuate section 42a, 42b in the illustrated embodiment has a width W₁ at the lower end portion 43b, 45b that is greater than a width W₂ at the upper end portion 43a, 45a. However, embodiments of the present invention are not limited to such a configuration. In some embodiments, each arcuate section 42a, 42b may have a width that is substantially constant between the upper end portions 43a, 45a and lower end portions 43b, 45b, respectively. In other embodiments, each arcuate section 42a, 42b in the illustrated embodiment may have a width W₁ at the lower end portion 43b, 45b that is less than a width W₂ at the upper end portion 43a, 45a. Also, the two sections 42a, 42b can be a single section or more than two cooperating sections, according to some embodiments of the present invention.

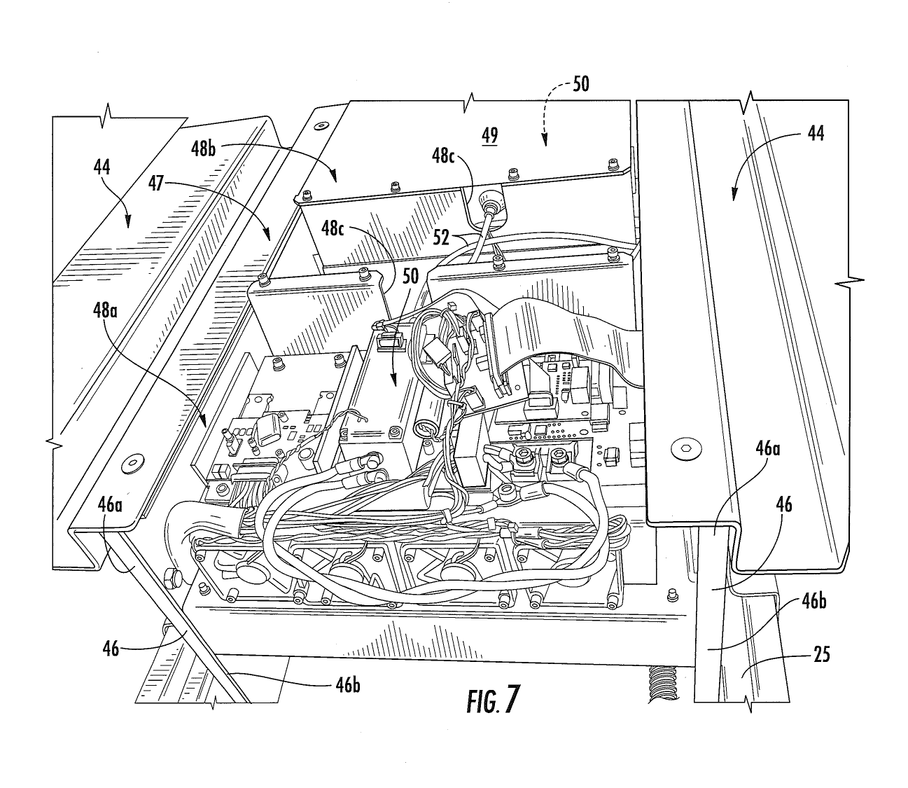

Still referring to FIG. 3, a laser housing compartment 47 is secured between the leg members 46 of the treatment frame 40. When the patient support surface 30 is installed on the base 20, the laser housing compartment 47 is positioned beneath the patient support surface 30. As the treatment frame 40 passes over a patient positioned on the patient support surface 30 during a treatment session, the compartment 47 passes beneath the patient support surface 30 within the cavity 23. The compartment 47 houses the laser devices 50 (FIG. 7) that provide respective laser beams to the respective laser collimators 60 (FIG. 1) secured to the treatment frame 40.

In some embodiments, each laser device 50 is a neodymium doped yttrium-aluminum-garnet laser configured to deliver a laser beam at a coherent, monochromatic wavelength of between 600 and 1400 nanometers (nm), typically about 1064 nm, in a continuous wave (CW) mode over a large spot area and in long pulses (e.g., pulse duration times of 1 minute, 2 minutes, 5 minutes and greater, etc.). Variables associated with laser therapy, according to embodiments of the present invention include radiation energy output of a laser device 50, the time of exposure, and the contact area, which define the energy density at the point of exposure of the biological tissue, and the radiation energy wavelength. Energy density is typically defined in terms of either milliwatt-seconds per square centimeter (“mW-s/cm²”) or joules per square centimeter (“J/cm²”), wherein one thousand milliwatts times one second equals one joule, and as indicated, the energy density represents the quantity of energy imparted to a specific area. Thus, the energy density is defined by the combination of the energy output of the laser beam, the time of exposure and the size of the area of biological tissue exposed to the laser beam, which is further a function of the distance of the laser beam from the surface exposed.

In some embodiments, the spot area produced by a collimator 60 may be at least about 10 cm². In other embodiments, the spot area produced by a collimator may be between about 12 cm² and 40 cm², and is typically about 28 cm². In some embodiments, each laser device 50 has a power greater than 10 W (e.g., 17.5 W). For a beam size of 12 cm², each laser 50 delivers a 600 mw/cm² CW laser beam over the 12 cm² area resulting in an energy delivery rate greater than or equal to about 420 Joules/min. Table 1 below summarizes values for laser device power, laser spot size, wavelength, energy delivery rate, and energy density, according to some embodiments of the present invention.

TABLE 1

| | Spot | | Energy | Energy |
|---|---|---|---|---|
| Power | Size | Wavelength | Delivery Rate | Density |
| (W) | (cm²) | (nm) | (Joules/min) | (mw/cm²) |
| 10 | 10 | 1064 | 600 | 1000 |
| 10 | 12 | 1064 | 600 | 833 |
| 10 | 28 | 1064 | 600 | 500 |
| 16.5 | 10 | 1064 | 990 | 1650 |
| 16.5 | 12 | 1064 | 990 | 1375 |
| 16.5 | 28 | 1064 | 990 | 589 |
| 20 | 10 | 1064 | 1200 | 2000 |
| 20 | 12 | 1064 | 1200 | 1667 |
| 20 | 28 | 1064 | 1200 | 714 |

The term “monochromatic” means the light is of substantially the same wavelength. The term “coherent” means the electromagnetic radiation waves making up the laser light have substantially the same direction, amplitude, and phase with respect to one another.

Treatment sessions using laser light from the laser devices 50 may deliver doses between 1 Joules/cm² to 400 Joules/cm². In some embodiments, patient tissue is exposed to at least 7 Joules/cm² of laser exposure and the exposure is maintained for a period of time sufficient to deliver a laser light dosage to the tissue of at least 1,500 Joules per treatment session, typically 10,000 Joules-20,000 Joules, per treatment session without patient discomfort or adverse effect. In certain applications, for example, when treating fever blisters, fibromyalgia, etc., laser light dosage to the tissue may exceed 32,000 Joules, and such treatments may be repeated daily.

In some embodiments, each laser device 50 may produce a laser beam with a wavelength of about 1064 nanometers (nm) in the near infrared region of the electromagnetic spectrum. A wavelength of 1064 nm is below the absorption range of melanin and at a low absorption rate of water and hemoglobin. A wavelength of 1064 nm is preferentially absorbed by mitochondrial chromophores, while absorption by skin, water, adipose tissue and hemoglobin is reduced. This allows a laser 50 to deliver a small dosage to many layers of tissue and much deeper penetration than conventional treatments.

Although Nd:Yag lasers have been described herein, embodiments of the present invention may utilize other types of lasers (e.g., near-infrared (NIR) lasers based upon the Cr⁴⁺-active ion, etc.).

The energy of the optical radiation is controlled and applied to produce an absorption rate in the irradiated tissue to minimize elevation of the average temperature of the irradiated tissue to a level above the basal body temperature and in no event to the extent the maximum absorption rate is great enough to convert the irradiated tissue into a collagenous substance. Infrared laser light is capable of imparting energy to cells deep within the tissue. For example, each laser device 50 may be capable of delivering laser light, via a respective collimator 60, into tissue up to a depth of about 4 inches or more. In the illustrated embodiment, the two collimators 60 are oriented to project two side-by-side, non-overlapping beams onto a patient.

Referring to FIG. 7, the illustrated laser housing compartment 47 includes two adjacent sections 48a, 48b. Each section 48a, 48b houses a respective laser device 50. In FIG. 7, a cover 49 overlies section 48b and the laser device 50 housed therein, and section 48a has the cover removed therefrom to illustrate the laser device 50 housed therewithin. Each illustrated section 48a, 48b includes an opening 48c through which a respective optical fiber cable 52 extends to connect each laser device 50 with a respective collimator 60. The optical fiber cables 52 are routed from each compartment section, 48a, 48b through the treatment frame 40 to the respective laser collimators 60.

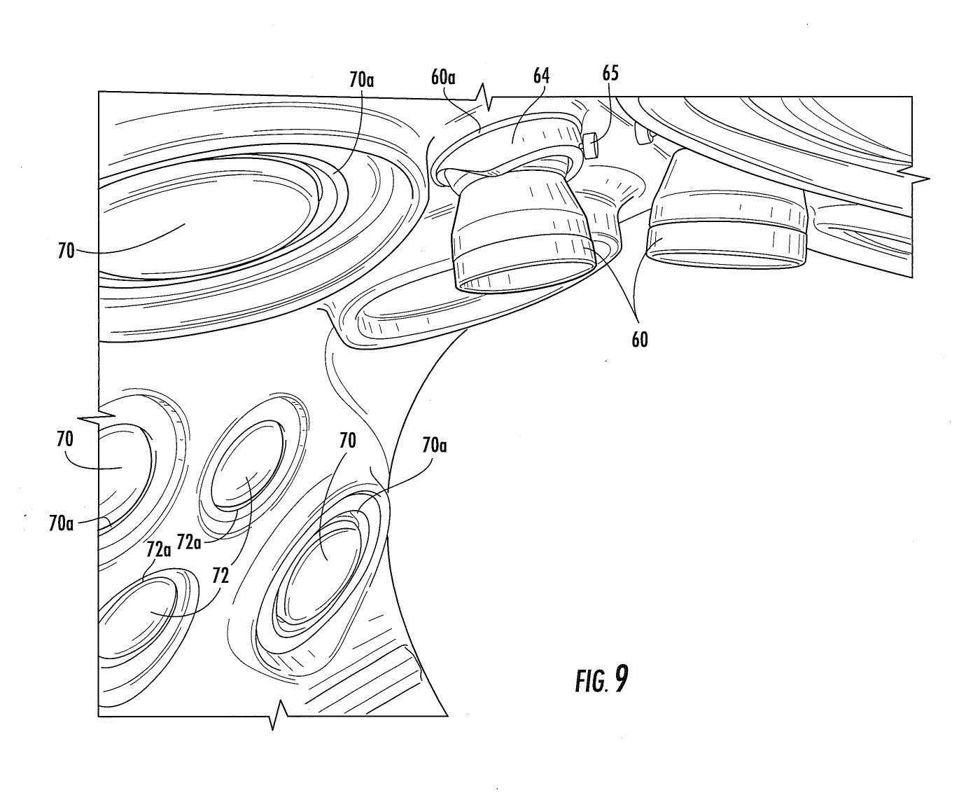

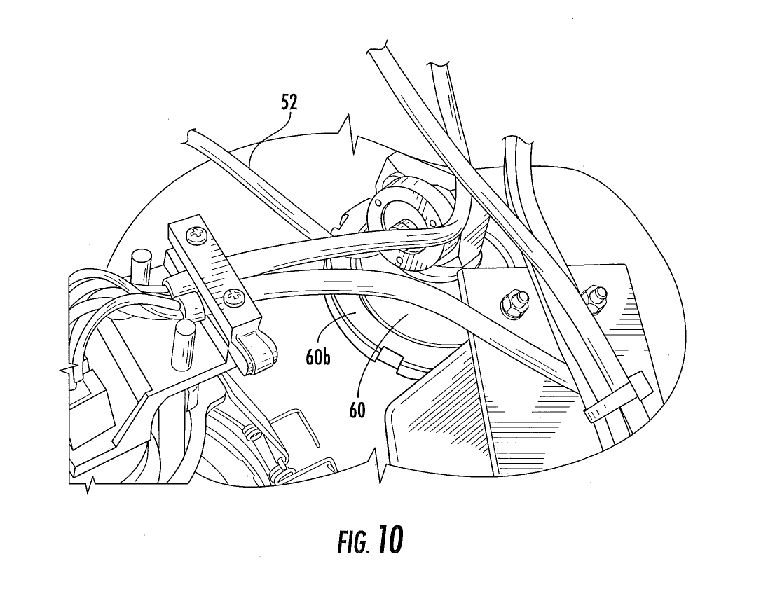

Referring back to FIG. 2, an opening 60a is formed through wall 41 of each section 42a, 42b of the treatment frame 40, and a respective laser collimator 60 extends through each opening 60a. Each collimator 60 is attached to the treatment frame 40 via a mounting bracket 60b secured to the back surface 41b of the wall 41, as illustrated in FIG. 10. Each collimator 60 extends outwardly from a front surface 41a of the treatment frame wall 41, as illustrated in FIG. 2, with the associated lens configured to provide the desired beam spot size. As illustrated in FIG. 9, each collimator 60 includes a collar 64 that is secured to the wall 41 and that permits adjustment/alignment of the collimator 60. A locking screw 65 on each collar 64 is provided to maintain each collimator 60 in a desired alignment, as would be understood by those skilled in the art.

In the illustrated embodiment, the pair of collimators 60 are in adjacent, spaced-apart relationship. Each laser collimator 60 is in optical communication with a respective laser device 50 via a respective optical fiber cable 52 and is configured to project a collimated laser beam onto the skin of a patient positioned on the patient support surface 30. Each collimator 60 increases the size of a laser beam carried by a respective optical fiber cable 52 while maintaining and enhancing the coherency and monochromacity of the laser beam. In some embodiments, each collimator 60 includes a series of lenses that convert a laser beam generated by a laser device 50 into a collimated beam of a predetermined cross-sectional area. For example, a first lens may be provided to initially enlarge the laser beam generated by the laser device, such as a 200 micron beam. A second lens may be provided to convert the enlarged beam into a column and project the enlarged beam onto a patient with a specific cross-sectional area (i.e., with the desired beam spot size).

In some embodiments of the present invention, each collimator 60 is configured to project a collimated laser beam having a cross-sectional area of at least approximately 10 cm² while maintaining the laser beam as generally coherent and monochromatic. In other embodiments, each collimator 60 is configured to project a collimated laser beam having a cross-sectional area of between about 12 cm² and about 40 cm², typically about 28 cm², while maintaining the laser beam as generally coherent and monochromatic. Embodiments of the present invention, however, are not limited to collimators 60 that produce a collimated beam of a particular cross-sectional area. Collimators 60 that can produce collimated beams of various sizes may be utilized.

In some embodiments, a laser beam projected onto the skin of a patient by a collimator 60 may be an infrared beam that is not visible to the naked eye. To indicate the location of an infrared beam, a marker beam of visible light may be projected by each collimator 60. FIG. 1 illustrates a visible marker beam 62 projected onto the mattress M by each respective collimator 60. A marker beam source (not illustrated), such as a 2.5 mw, 535 nm diode, is in optical communication with each collimator 60. Each collimator 60 is configured to project a visible marker beam generated by the marker beam source simultaneously with the laser treatment light, onto the skin of a patient positioned on the patient support surface 30 (or positioned on a mattress M on the patient support surface 30).

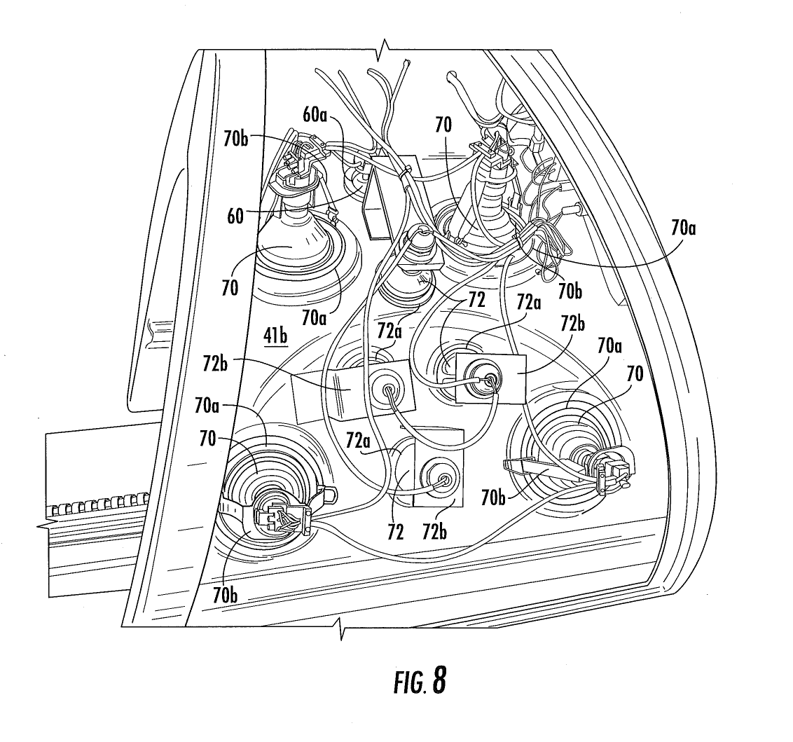

According to embodiments of the present invention, the treatment frame 40 may include one or more heat sources for providing warmth to a patient positioned on the patient support surface 30. In the illustrated embodiment, a plurality of first and second openings 70a, 72a (FIG. 8) are formed through wall 41 of the treatment frame 40 and a respective plurality of first and second heat lamps 70, 72 extend therethrough and serve as heat sources. Because of the heat generated by the heat lamps 70, 72, it may be undesirable for contact to occur between the heat lamps 70, 72 and a patient. As such, as illustrated in FIG. 2, the heat lamps 70, 72 are substantially flush with the front surface 41a of treatment frame wall 41. In other embodiments the heat lamps 70, 72 may be slightly recessed from the front surface 41a of treatment frame wall 41.

The first heat lamps 70 in the illustrated embodiment have larger diameters than the second heat lamps 72 and are configured to provide warmth to a greater area of a patient's body than the second heat lamps 72. In some embodiments, the first heat lamps 70 are red heat lamps and the second heat lamps 72 are blue heat lamps. The first and second heat lamps 70, 72 may be used in various combinations to provide warmth to a patient.

FIG. 8 is a side view of arcuate section 42b of the illustrated treatment frame 40 with an outer cover removed. Each arcuate section 42a, 42b serves as a housing for the heat lamps 70, 72, and for the various electrical wiring connected to the heat lamps 70, 72. Each arcuate section 42a, 42b also serves as a housing for a respective laser collimator 60 and the optical fiber cabling attached to each.

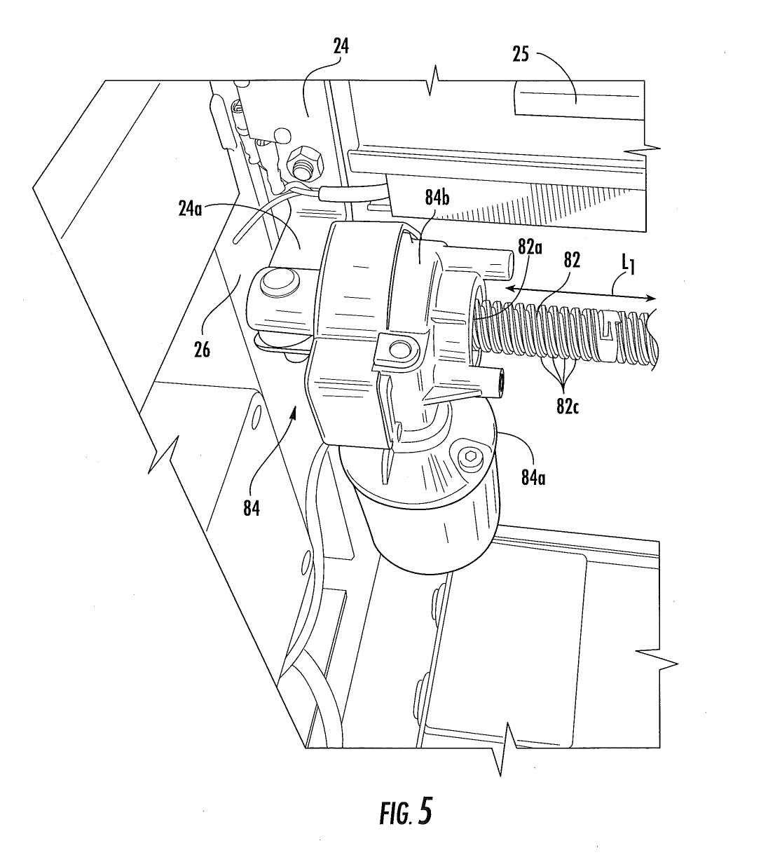

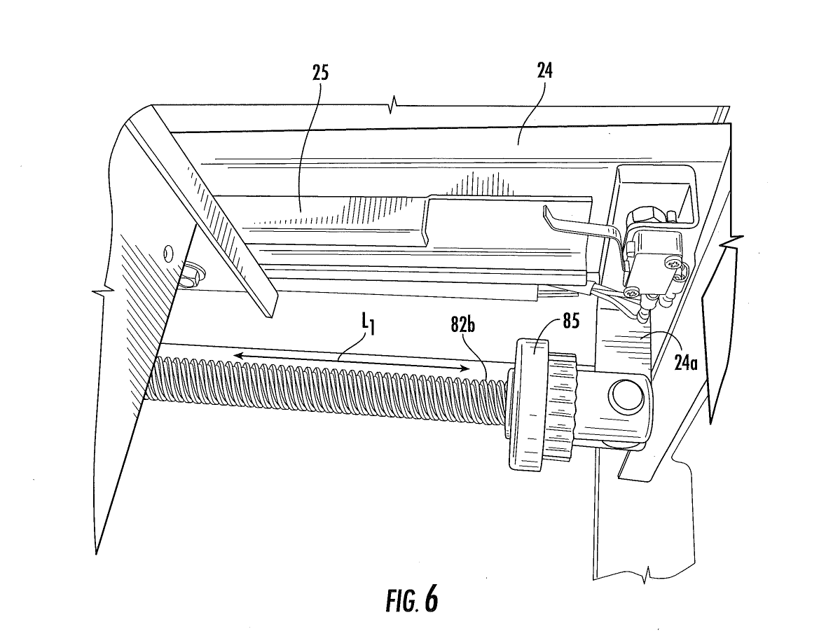

Referring to FIGS. 3-6, the treatment frame 40 is movably secured to the base 20 and is movable relative to the base 20 along the longitudinal direction L₁ via a drive system 80 located within the base cavity 23. In the illustrated embodiment, the drive system 80 includes an elongated threaded drive shaft 82 that extends between the opposite end walls 22 of the base 20 and that is supported by the frame 24, as described below. As illustrated in FIG. 5, a bracket 24a extends outwardly from the frame 24 to support a motor assembly 84. One end 82a of the drive shaft 82 is rotationally secured to the motor assembly 84, as will be described below. The opposite end 82b of the drive shaft 82 is rotationally mounted within a bearing housing 85 (FIG. 6) that is mounted to the frame 24 via a bracket 24a extending outwardly therefrom.

As illustrated in FIG. 5, the motor assembly 84 includes an electric motor 84a and a gearbox 84b. An end 82a of the drive shaft 82 is rotatably mounted within the gearbox 84b and the threads 82c of the drive shaft 82 are intermeshed with a rotational gear (not shown) within the gearbox 84b, as would be understood by those skilled in the art. During operation, the electric motor 84a rotates an output shaft (not shown) which, in turn causes rotation of the gear intermeshed with the end 82a of the drive shaft 82, which in turn causes rotation of the drive shaft 82, as would be understood by those skilled in the art.

A gear (not shown) is attached to the treatment frame 40 and is intermeshed with the threads 82c of the drive shaft 82. Rotation of the drive shaft 82 via motor 84 produces linear motion of the treatment frame 40 along the longitudinal direction L₁, as would be understood by those skilled in the art. During a treatment session, the treatment frame 40 can be substantially continuously moved along the longitudinal direction L₁. The drive system 80 is configured to move the treatment frame 40 at a constant speed within a range of between about 0.05 inch per second and about one inch per second. However, other speeds may be obtainable. In addition, the treatment frame 40 can be held in one or more locations for a desired period of time during a treatment session.

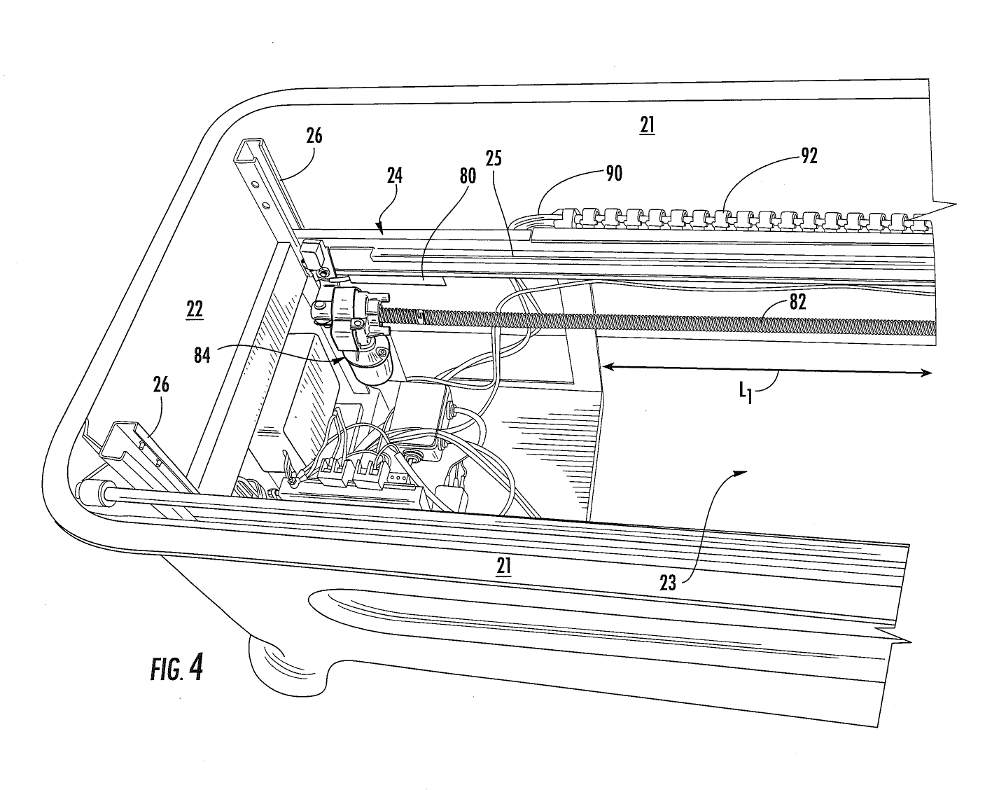

As illustrated in FIGS. 3 and 4, cables 90 for providing power to the various components supported by the treatment frame (i.e., heat lamps 70, 72; laser devices 50; control panel 100) are configured to move with the treatment frame 40 along the longitudinal direction L₁. A cable chain 92 surrounds the various cables 90 and is configured to coil and uncoil with movement of the treatment frame 40 so as to guide and protect the cables 90. Cable chains are well known in the art and need not be described further herein.

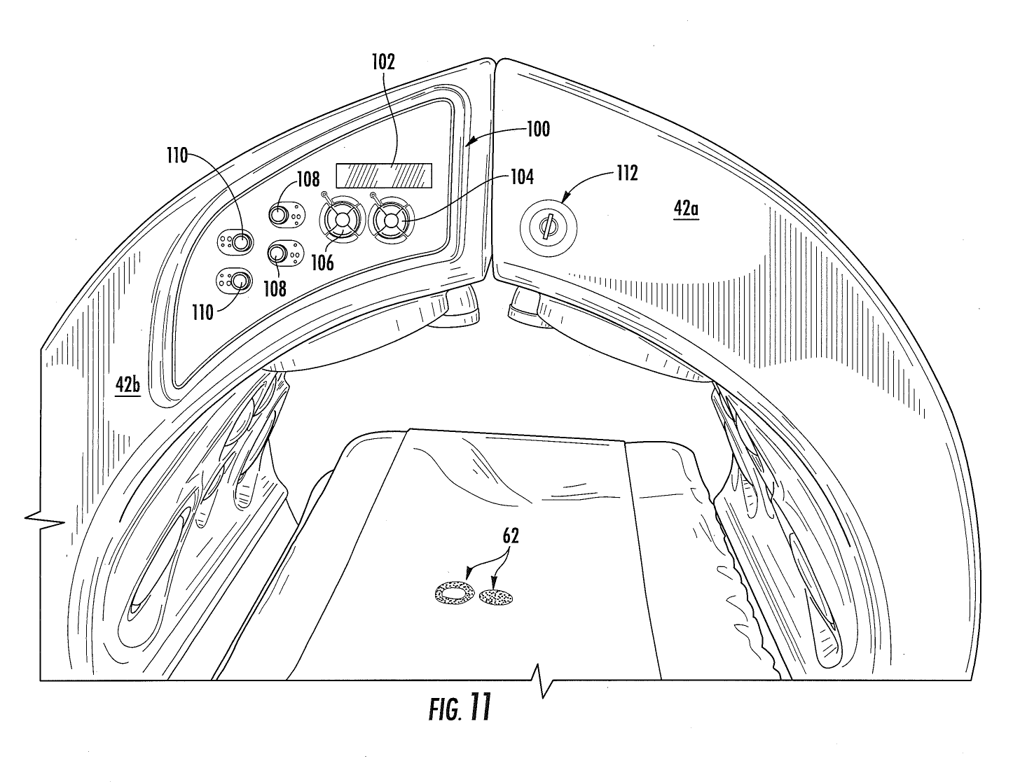

The laser therapy apparatus 10 also includes a controller or processor (not shown) having a control panel 100 (FIG. 11) for controlling various operations of the apparatus 10, including travel speed of the treatment frame 40, travel time of the treatment frame 40, output of the laser devices 50, etc. The processor can be a conventional programmable controller and/or can include an application specific integrated circuit (ASIC) configured to control operation of the laser therapy apparatus 10, or a general microprocessor or controller (e.g. computer). The processor contains a control program (firmware, software, etc.) that dictates the operation of laser therapy apparatus 10. In addition, the processor may optionally include stored protocols (time, energy, spot size) for different laser treatments. These stored programs may be selected from the control panel 100. Also, in some embodiments, a foot pedal (not shown) may be connected to the laser devices 50 for allowing a healthcare provider, technician, etc., to control the laser devices 50 remotely from the control panel 100, if desired. In some embodiments, a clock or timer may be associated with the processor to limit energy applied during a treatment session.

The illustrated control panel 100 (FIG. 11) includes a display screen 102 that displays various operational parameters and conditions. Information displayed via the display screen 102 may include travel speed of the treatment frame 40, travel time of the treatment frame 40, power density of laser beams projected by the collimators 60, etc. User control 104 is a four-way rocker switch that is used to set treatment times and other parameters, such as energy or power density of each laser device 50. User control 106 is a four-way rocker switch that is used to set travel distance and travel speeds of the treatment frame 40 relative to the base 20. User controls 108 are switches that are used to control operation of the blue heat lamps 72 and user controls 110 are switches that are used to control operation of the red heat lamps 70. A “kill switch” 112 is provided that can be utilized by an operator and a patient to stop movement of the treatment frame 40, shut off the heat lamps 70, 72, and shut off the laser devices 50 during a treatment session. In the illustrated embodiment, the kill switch 112 is also a keyed lock-out switch that prevents unauthorized operation of the apparatus 10.

In the illustrated embodiment, the control panel 100 is located on treatment frame section 42b and the kill switch 112 is located on the treatment frame section 42a. However, embodiments of the present invention are not limited to this arrangement. In some embodiments, the control panel 100 and kill switch 112 may be located on the same treatment frame section 42a, 42b. in other embodiments, the control panel 100 may be located on treatment frame section 42a and the kill switch 112 may be located on treatment frame section 42b.

Utilizing the laser therapy apparatus 10 described above, a method of treating selected tissue of a patient includes moving the treatment frame 40 relative to the base 20 upon which a patient is supported at a substantially constant speed (e.g., between about 0.05 inch per second and about one inch per second, etc.), and projecting a coherent and monochromatic laser beam (e.g., having a wavelength of between 600 and 1400 nanometers) having a delivery rate of at least 420 Joules/min, a power density of at least 500 mw/cm², an energy density delivery of at least 1,500 Joules/cm², and a cross-sectional area of at least 10 cm² from the treatment frame onto the skin of the patient such that the selected tissue is exposed to a laser light dosage of at least 1,500 Joules during a treatment session. In some embodiments, the patient is warmed during treatment via the first and/or second heat lamps 70, 72. In some embodiments, each collimator 60 is configured to project a collimated laser beam having a cross-sectional area of at least 28 cm².

Various laser treatments, which may be implemented via the treatment apparatus 10, are described in U.S. Patent Application Publication No. 2007/0162093, which is incorporated herein by reference in its entirety. Selected tissue that may be treated via the laser therapy apparatus 10 includes: tissue physiologically linked to peripheral neuropathy, reflex sympathetic dystrophy, trigeminal neuralgia, migraine headaches, stroke, concussions, plantar fascititis, radiculophthy, peripheral neuropathy, sciatica, traumatic nerve injury, diabetic nerve, or restless leg syndrome; tissue physiologically linked to post-operative healing, decubitis wound sores, burns, stasis ulcers, allergic rashes, or insect bites; tissue physiologically linked to spinal pain from herniated disc, spinal pain from herniated bulging disc, back pain from musculoskeletal strain, reflex symphatic dystrophy, or fibromyalgia; tissue physiologically linked to herpes, acquired immune deficiency syndrome (AIDS), multiple sclerosis, psoriasis, rheumatoid diseases, chronic fatigue syndrome, Parkinson's disease, or lupus; tissue physiologically linked to fibromyalgia or costochondritis; tissue physiologically linked to stroke, closed head injury, or spinal cord injury; tissue physiologically linked to coronary artery disease or peripheral vascular disease; tissue physiologically linked to viral flu syndromes or autoimmune diseases; tissue physiologically linked to diabetes or ALS; musculo-skeletal tissue; neurological tissue; wound tissue; and spinal tissue or spinal fluid.

According to some embodiments of the present invention, the following steps are performed using the apparatus 10 described above after a patient is positioned on the patient support surface 30 (e.g., on the mattress M overlying the patient support surface 30) with the desired tissue to be tissue exposed and up.
Step 1. Using control panel controls, move the treatment frame to position the laser collimators to one end of the tissue to be treated. Use the marker beams for alignment. Aim lens of collimator at selected tissue.
Step 2. Set the first travel limit switch to establish this as the beginning point of the area to be treated. This sets a stop/return point so the laser beam cannot travel beyond that point.
Step 3. Move treatment frame to other end of area to be treated. Move the second limit switch to establish the second stop/return point.
Step 4. Set power levels of laser 1 and laser 2.
Step 5. Set time—minutes.
Step 6. Set travel speed of treatment frame.
Step 7. Make sure therapist and patient have on safety glasses.
Step 8. Engage foot peddle to activate treatment.
Step 9. Upon completion remove travel stops to the outer limits and return treatment frame to home position (end). Patient is free to exit the apparatus.
   Generally, the apparatus 10 delivers approximately 33,000 mw or 33 w continually. This enables a patient to be treated substantially faster than with conventional devices. Moreover, the apparatus 10 enables a physician to keep the patient comfortable with the heat sources. In addition, the patient support surface 30 and mattress M allow for comfortable positioning of patients.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the teachings and advantages of this invention. Accordingly, all such modifications are intended to be included within the scope of this invention as defined in the claims. The invention is defined by the following claims, with equivalents of the claims to be included therein.

That which is claimed is:

1. A laser therapy apparatus, comprising:
an elongated base that defines a longitudinal direction;
a patient support surface attached to the base;
a treatment frame movably secured to the base and movable relative to the base along the longitudinal direction, wherein the treatment frame extends over the patient support surface and is configured to pass over a patient positioned on the patient support surface;
a laser device configured to produce a laser beam; and
a collimator attached to the treatment frame and in optical communication with the laser device, wherein the collimator is configured to receive a laser beam from the laser device, collimate the laser beam to a cross-section area of at least approximately 10 cm² while maintaining the laser beam as generally coherent and monochromatic, and project the collimated laser beam onto the skin of a patient positioned on the patient support surface.

2. The apparatus of claim 1, wherein the laser device is a neodymium doped yttrium-aluminum-garnet laser configured to deliver a laser beam at a wavelength of about 1064 nanometers (nm).

3. The apparatus of claim 1, wherein the laser device produces a generally coherent and monochromatic laser beam having an energy delivery rate of at least 420 Joules/min, a power density of at least 500 mw/cm², and an energy density delivery of at least 1,500 Joules/cm².

4. The apparatus of claim 1, comprising:
a pair of laser devices; and
a pair of collimators attached to the treatment frame in adjacent, spaced-apart relationship, wherein each collimator is in optical communication with a respective laser device, and wherein the collimators are configured to project two side-by-side, non-overlapping collimated beams onto the skin of a patient positioned on the patient support surface.

5. The apparatus of claim 1, wherein the collimator is configured to collimate the laser beam to a cross-section area of at least approximately 28 cm².

6. The apparatus of claim 1, wherein the treatment frame comprises at least one heat source configured to warm a patient positioned on the patient support surface.

7. The apparatus of claim 6, wherein the at least one heat source comprises a plurality of heat lamps.

8. The apparatus of claim 1, wherein the collimator is in optical communication with the laser device via an optical fiber cable.

9. The apparatus of claim 1, wherein the treatment frame includes a compartment positioned beneath the patient support surface, and wherein the laser device is housed within the compartment.

10. The apparatus of claim 1, wherein the base comprises opposite elongated side walls and opposite end walls that define a cavity, and wherein the treatment frame is movably secured to a pair of guides secured to the base within the cavity.

11. The apparatus of claim 1, wherein the treatment frame has an arcuate configuration that extends from one side wall to the other side wall.

12. The apparatus of claim 10, further comprising a drive system located within the base cavity that is configured to move the treatment frame relative to the base along the longitudinal direction, wherein the drive system comprises:
an elongated threaded drive shaft that extends between the opposite end walls of the base;
a motor operably connected to the drive shaft and configured to rotate the drive shaft; and
a gear attached to the treatment frame that is intermeshed with the threaded drive shaft for producing linear motion of the treatment frame along the longitudinal direction upon rotation of the drive shaft.

13. The apparatus of claim 12, wherein the drive system is configured to move the treatment frame at a constant speed within a range of between about 0.05 inch per second and about one inch per second.

14. The apparatus of claim 1, further comprising a marker beam source in optical communication with the collimator, wherein the collimator is configured to project a visible marker beam generated by the marker beam source onto the skin of a patient positioned on the patient support surface that indicates the location of the collimated laser beam on the skin of the patient.

15. The apparatus of claim 1, wherein the treatment frame includes a control panel for controlling operations of the apparatus.

16. A laser therapy apparatus, comprising:
an elongated base that defines a longitudinal direction;
a patient support surface attached to the base;
a treatment frame movably secured to the base and movable relative to the base along the longitudinal direction, wherein the treatment frame extends over the patient support surface and is configured to pass over a patient positioned on the patient support surface, and wherein the treatment frame includes a compartment positioned beneath the patient support surface;
at least one heat source attached to the treatment frame that is configured to warm a patient positioned on the patient support surface;
a pair of laser devices housed within the compartment, wherein each laser device is configured to produce a respective laser beam; and
a pair of collimators attached to the treatment frame in adjacent, spaced-apart relationship, wherein each collimator is in optical communication with a respective laser device via a respective optical fiber cable, wherein each collimator is configured to receive a laser beam from a laser device, collimate the laser beam to a cross-section area of at least approximately 10 cm² while maintaining the laser beam as generally coherent and monochromatic, and project the collimated laser beam onto the skin of a patient positioned on the patient support surface.

17. The apparatus of claim 16, wherein each laser device is a neodymium doped yttrium-aluminum-garnet laser configured to deliver a laser beam at a wavelength of about 1064 nanometers (nm).

18. The apparatus of claim 16, wherein each laser device produces a generally coherent and monochromatic laser beam having an energy delivery rate of at least 420 Joules/min, a power density of at least 500 mw/cm², and an energy density delivery of at least 1,500 Joules/cm².

19. The apparatus of claim 16, wherein each collimator is configured to collimate a respective laser beam to a cross-section area of at least approximately 28 cm².

20. The apparatus of claim 16, wherein the at least one heat source comprises a plurality of heat lamps.

21. The apparatus of claim 16, wherein the base comprises opposite elongated side walls and opposite end walls that define a cavity, and wherein the treatment frame is movably secured to a pair of guides secured to the base within the cavity.

22. The apparatus of claim 21, further comprising a drive system located within the base cavity that is configured to move the treatment frame relative to the base along the longitudinal direction, wherein the drive system comprises:
an elongated threaded drive shaft that extends between the opposite end walls of the base;
a motor operably connected to the drive shaft and configured to rotate the drive shaft; and
a gear attached to the treatment frame that is intermeshed with the threaded drive shaft for producing linear motion of the treatment frame along the longitudinal direction upon rotation of the drive shaft.

23. The apparatus of claim 22, wherein the drive system is configured to move the treatment frame at a constant speed within a range of between about 0.05 inch per second and about one inch per second.

24. The apparatus of claim 16, further comprising a marker beam source in optical communication with each collimator, wherein each collimator is configured to project a visible marker beam generated by the marker beam source onto the skin of a patient positioned on the patient support surface that indicates the location of the collimated laser beam on the skin of the patient.

25. The apparatus of claim 16, wherein the treatment frame includes a control panel having controls for controlling movement of the treatment frame relative to the base, for controlling operation of the laser devices and collimators, and for controlling operation of the at least one heat source.

26. A method of treating selected tissue of a patient, comprising:
moving a treatment frame relative to a base upon which a patient is supported at a constant speed; and
projecting a coherent and monochromatic laser beam having a delivery rate of at least 420 Joules/min, a power density of at least 500 mw/cm², an energy density delivery of at least 1,500 Joules/cm², and a cross-sectional area of at least 10 cm² from the treatment frame onto the skin of the patient such that the selected tissue is exposed to a laser light dosage of at least 1,500 Joules during a treatment session.

27. The method of claim 26, further comprising warming the patient via at least one heat source on the treatment frame simultaneously with projecting the collimated laser beam.

28. The method of claim 26, wherein moving a treatment frame relative to a base upon which a patient is supported at a constant speed comprises moving the treatment frame at a constant speed within a range of between about 0.05 inch per second and about one inch per second.

29. The method of claim 26, wherein the collimated laser beam has a wavelength of about 1064 nanometers (nm).

30. The method of claim 26, comprising projecting a collimated laser beam with a cross-sectional area of at least 28 cm².

31. The method of claim 26, wherein the selected tissue is tissue physiologically linked to peripheral neuropathy, reflex sympathetic dystrophy, trigeminal neuralgia, migraine headaches, stroke, concussions, plantar fascititis, radiculophthy, peripheral neuropathy, sciatica, traumatic nerve injury, diabetic nerve, or restless leg syndrome.

32. The method of claim 26, wherein the selected tissue is tissue physiologically linked to post-operative healing, decubitis wound sores, burns, stasis ulcers, allergic rashes, or insect bites.

33. The method of claim 26, wherein the selected tissue is tissue physiologically linked to spinal pain from herniated disc, spinal pain from herniated bulging disc, back pain from musculoskeletal strain, reflex symphatic dystrophy, or fibromyalgia.

34. The method of claim 26, wherein the selected tissue is tissue physiologically linked to herpes, acquired immune deficiency syndrome (AIDS), multiple sclerosis, psoriasis, rheumatoid diseases, chronic fatigue syndrome, Parkinson's disease, or lupus.

35. The method of claim 26, wherein the selected tissue is tissue physiologically linked to fibromyalgia or costochondritis.

36. The method of claim 26, wherein the selected tissue is tissue physiologically linked to stroke, closed head injury, or spinal cord injury.

37. The method of claim 26, wherein the selected tissue is tissue physiologically linked to coronary artery disease or peripheral vascular disease.

38. The method of claim 26, wherein the selected tissue is tissue physiologically linked to viral flu syndromes or autoimmune diseases.

39. The method of claim 26, wherein the selected tissue is tissue physiologically linked to diabetes or ALS.

40. The method of claim 26, wherein the selected tissue is musculo-skeletal tissue.

41. The method of claim 26, wherein the selected tissue is neurological tissue.

42. The method of claim 26, wherein the selected tissue is wound tissue.

43. The method of claim 26, wherein the selected tissue is spinal tissue or spinal fluid.
